SEQUENCE LISTING (1 sequence)

SEQ ID NO: 1 - protein, 887 aa, Homo sapiens
MGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

(19) United States
(12) Patent Application Publication
(10) Pub. No.: US 2020/0289613 A1
(43) Pub. Date: Sep. 17, 2020

(54) METHODS OF TREATING METASTATIC CANCERS USING AXL DECOY RECEPTORS

(71) Applicant: Aravive Biologics, Inc., Houston, TX (US)

(72) Inventors: Gail McIntyre, Lansdale, PA (US); David Prohaska, Madison, WI (US); Ray Tabibiazar, Chattanooga, TN (US)

(21) Appl. No.: 16/761,246

(22) PCT Filed: Nov. 5, 2018
(86) PCT No.: PCT/US2018/059218

§ 371 Date: May 2, 2020

Related U.S. Application Data
(60) Provisional application No. 62/681,944, filed on Jun. 7, 2018.
(60) Provisional application No. 62/618,916, filed on Jan. 18, 2018.
(60) Provisional application No. 62/581,671, filed on Nov. 4, 2017.

(51) Int. Cl. A61K 38/17; A61K 38/45; A61P 35/04; C07K 14/705

(52) U.S. Cl. CPC A61K 38/177; A61K 38/45; A61K 45/06; C07K 14/705; C12Y 207/10001; A61P 35/04

(57) ABSTRACT

Compositions and methods are provided for treating a human metastatic cancer in a mammal by administering a therapeutic dose of a pharmaceutical composition that inhibits AXL protein activity, for example by inhibition of the binding interaction between AXL and its ligand GAS6.

### RELATED PATENT APPLICATIONS

This application claims benefit of U.S. Provisional Application No. 62/581,671, filed on Nov. 4, 2017; U.S. Provisional Application No. 62/618,916, filed on Jan. 18, 2018; and U.S. Provisional Application No. 62/681,944, filed on Jun. 7, 2018, each incorporated in its entirety by reference herein.

### SEQUENCE LISTING

The instant application contains a Sequence Listing in the form of a “paper copy” (PDF File) and a file containing the referenced sequences (SEQ D NO: 1) in computer readable form (ST25 format text file) which is submitted herein. The Sequence Listing is shown using standard three letter code for amino acids, as defined in 37 C.F.R. 1.822.

### STATEMENT REGARDING FEDERALLY SPONSORED RESEARCH OR DEVELOPMENT

This work was supported by Cancer Prevention & Research Institute of Texas, New Company Product Development Award DP150127. The State of Texas, USA, may have rights in any patent issuing on this application.

### TECHNICAL FIELD

Cancer is group of diseases involving abnormal cell growth with the potential to spread or invade other parts of the body. Abnormal growths that form a discrete tumor mass, i.e., do not contain cysts or liquid areas, are defined as solid tumors. Solid tumors may be benign (not cancer), or malignant (cancer). Different types of solid tumors are named for the type of cells that form them. Examples of solid tumors are sarcomas, carcinomas, and lymphomas. Cancers derived from either of the two blood cell linages, myeloid and lymphoid, are defined as hematological malignancies. Such malignancies are also referred to as blood cancers or liquid tumors. Examples of liquid tumors include multiple myeloma, acute leukemias (e.g., 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (e.g., chronic myelocytic (granulocytic) leukemia, chronic myelogenous leukemia, and chronic lymphocytic leukemia), polycythemia vera, lymphoma, Hodgkin's lymphoma, non-Hodgkin's lymphoma (indolent and high grade forms), Waldenstrom's macroglobulinemia, heavy chain disease, myelodysplastic syndrome, hairy cell leukemia and myelodysplasia.

Treatments available to cancer patients are heavily dependent on combination therapy including surgery, cytoreductive therapy and cytotoxic chemotherapy. Unfortunately, while effective in some cases, the normal tissue side effects often manifest as dose limiting toxicities and prevent tumor eradication. And even when the side effects of these various therapies can be managed, long-lasting responses are often elusive; particularly, for therapeutically refractive metastatic diseases.

The concept of targeted therapies, i.e., target a specific molecule or signaling pathway, was developed to tackle these problems and reduce normal tissue side effects. However, most patients either fail to exhibit a response to these targeted therapies or respond and then quickly relapse. Thus, there is a need for more effective targeted therapies that can be used in combination with current standards of care to address metastatic and treatment refractory disease.

Therapeutic efforts in cancer prevention and treatment are being focused at the level of signaling pathways or selective modulatory proteins. Protein kinase activities, calcium homeostasis, and oncoprotein activation are driving signals and therefore may be key regulatory sites for therapeutic intervention. The AXL receptor and its activating ligand, growth arrest—specific 6 (GAS6), are important drivers of metastasis and therapeutic resistance in human cancers. AXL belongs to the TAM family of receptor tyrosine kinases, which include Tyro3 (or SKY), AXL, and MER (O'Bryan, JR, Molecular and Cellular Biology, 5016-5031, 1991). GAS6 is the common ligand for all three receptors. In contrast to other TAM family members that have redundant ligands for signaling, AXL's only known ligand is GAS6 (with a very high natural binding affinity). Over-expression and activation of the GAS6-AXL signal transduction pathway has been found to be important in a wide variety of human tumors including renal, pancreatic, breast, lung, ovarian and prostate cancer (Rankin, EK, PNAS, 13373-13378, 2014). AXL overexpression in highly metastatic cancers is associated with poor prognosis, aggressive tumor behavior, and resistance to therapy. Studies show that in solid tumors, activation of the GAS6-AXL signaling pathway promotes tumor invasion and metastasis, as well as the development of resistance to commonly used chemotherapeutic agents. Given the critical roles that GAS6 and AXL play in advanced and refractory cancers, this signaling axis represents an attractive target for therapeutic intervention. Unfortunately, an unusually strong binding affinity between GAS6 and AXL of ˜30 μM has made the development of competitive antagonists challenging.

The AXL receptor contains two distinct GAS6 binding epitopes; a high affinity site on its N-terminal immunoglobulin-like (Ig) domain and a low affinity site on the second Ig domain. The present inventors have engineered the major site on AXL Ig1 using a combination of rational and combinatorial protein engineering methods to provide long-half-life AXL “decoy receptors” that bind GAS6 with higher affinity than endogenous AXL, effectively sequestering GAS6 and abrogating AXL signaling. These decoy receptors reduce invasion/migration of highly metastatic cells in vitro and inhibit metastatic disease in aggressive preclinical models of human pancreatic, renal, breast, and ovarian cancers, and exhibit a benign safety profile. When directly compared in nonclinical models with the most advanced anti-AXL small molecules currently developed in the clinic, the decoy receptors achieved superior antitumor efficacy while displaying no toxicity in pharmacology studies. And importantly, while the majority of oncology agents are required to initiate clinical studies in a cancer patient population due to significant toxicity associated with efficacious doses caused by both on-target and off-target effects, the decoy receptors engineered by the present inventors are not a cytotoxic drug.

Patent documents Ser. Nos. 13/554,954; 13/595,936; 13/714,875; 13/950,111; 14/712,731; 14/650,852; 14/650,854; 14/910,565; US2011/022125; US2013/056435; US2012/069841; US2013/074809; US2013/074786; US2013/074796; US2015/0315553 are herein specifically incorporated by reference for all teachings.

### DISCLOSURE OF THE INVENTION

In one aspect, the present invention provides methods for the treatment of a proliferative disease, e.g, a human metastatic cancer, comprising the administration of a soluble AXL polypeptide according to a regimen determined to achieve prolonged overall survival as compared to control.

In some embodiments, the proliferative disease is a cancer selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias. In some embodiments, the cancer is ovarian cancer. In some embodiments, the cancer is a breast cancer.

In some embodiments, the cancer is a cancer that overexpresses the biomarker GAS6 and/or AXL. In some embodiments, the cancer is a recurrent cancer. In some embodiments, the cancer is a human metastatic cancer resistant to standard therapies. In some embodiments, the human metastatic cancer is a chemoresistant cancer. In some embodiments, the human metastatic cancer is a platinum resistant cancer.

In another aspect, the present invention provides methods for the treatment of human metastatic cancers, comprising the administration of a soluble AXL polypeptide that lacks the AXL transmembrane domain and has at least one mutation relative to wild-type AXL that increases affinity of the AXL polypeptide binding to GAS6 compared to wild-type AXL, in combination with a second therapy selected from the group consisting of: small molecule kinase inhibitor targeted therapy, surgery, cytoreductive therapy, cytotoxic chemotherapy, and immunotherapy.

In some embodiments, the second therapy is cytoreductive therapy and the combination may increase the therapeutic index of the cytoreductive therapy. In some embodiments, the cytoreductive therapy may act in a DNA repair pathway. In some embodiments, the cytoreductive therapy is radiation therapy. In some embodiments, the combination may be synergistic.

In some embodiments, the second therapy is a chemotherapeutic agent is selected from the group consisting of: daunorubicin, adriamycin (doxorubicin), epirubicin, idarubicin, anamycin, MEN 10755, etoposide, teniposide, vinblastine, vincristine, vinorelbine (NAVELBINE); vindesine, vindoline, vincamine, mechlorethamine, cyclophosphamide, melphalan (L-sarcolysin), carmustine (BCNU), lomustine (CCNU), semustine (methyl-CCNU), streptozocin, chlorozotocin, cytarabine (CYTOSAR-U), cytosine arabinoside, fluorouracil (5-FU), floxuridine (FUdR), thioguanine (6-thioguanine), mercaptopurine (6-MP), pentostatin, fluorouracil (5-FU), methotrexate, 10-propargyl-5,8-dideazafolate (PDDF, CB3717), 5,8-dideazatetrahydrofolic acid (DDATHF), leucovorin, cisplatin (cis-DDP), carboplatin, oxaliplatin, hydroxyurea, gemcitabine, and N-methylhydrazine. In some embodiments, the combination may be synergistic.

In some embodiments, the second therapy will comprise immunotherapy selected from, but not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; wherein the combination therapy provides increased effector cell killing of tumor cells, i.e., a synergy exists between the soluble AXL polypeptide and the immunotherapy when co-administered.

In some embodiments, the second therapy will comprise administration of a poly (ADP-ribose) polymerase inhibitor (PARP) inhibitor. In some embodiments, the PARP inhibitor is selected from the group consisting of ABT-767, AZD 2461, BGB-290, BGP 15, CEP 9722, E7016, E7449, fluzoparib, INO1001, JPI 289, MP 124, niraparib, olaparib, ONO2231, rucaparib, SC 101914, talazoparib, veliparib, WW 46, or salts or derivatives thereof. olaparib, rucaparib, niraparib, talazoparib and veliparib. In some embodiments, the combination may be synergistic.

In some embodiments, the method of treatment will comprise the administration of a soluble AXL variant polypeptide in combination with pegylated liposomal doxorubicin (PLD). In some embodiments, the method of treatment will comprise the administration of a soluble AXL variant polypeptide in combination with paclitaxel. In some embodiments, the combination may be synergistic.

In some embodiments, the soluble AXL polypeptide is a soluble AXL variant polypeptide, wherein said soluble AXL variant polypeptide lacks the AXL transmembrane domain, lacks a functional fibronectin (FN) domain, has one or more Ig1 domain, has one or more Ig2 domain, and wherein said AXL variant polypeptide exhibits increased affinity of the AXL variant polypeptide binding to GAS6 compared to wild-type AXL.

In some embodiments, the soluble AXL polypeptide is a soluble AXL variant polypeptide, wherein said soluble AXL variant polypeptide lacks the AXL transmembrane domain, lacks a functional fibronectin (FN) domain, has one Ig1 domain, lacks a functional Ig2 domain and wherein said AXL variant polypeptide exhibits increased affinity of the AXL variant polypeptide binding to GAS6 compared to wild-type AXL.

In some embodiments, the AXL variant polypeptide is a fusion protein comprising an Fc domain. In some embodiments, the variant polypeptide lacks the AXL intracellular domain. In some embodiments, the soluble AXL variant polypeptide further lacks a functional fibronectin (FN) domain and wherein said variant polypeptide exhibits increased affinity of the polypeptide binding to GAS6. In some embodiments, the soluble AXL variant polypeptide comprises at least one amino acid modification relative to the wild-type AXL sequence.

In some embodiments, the soluble AXL variant polypeptide comprises at least one amino acid modification within a region selected from the group consisting of 1) between 15-50, 2) between 60-120, and 3) between 125-135 of the wild-type AXL sequence (SEQ ID NO:1).

In some embodiments, the soluble AXL variant polypeptide comprises at least one amino acid modification at position 19, 23, 26, 27, 32, 33, 38, 44, 61, 65, 72, 74, 78, 79, 86, 87, 88, 90, 92, 97, 98, 105, 109, 112, 113, 116, 118, or 127 of the wild-type AXL sequence (SEQ ID NO: 1) or a combination thereof.

In some embodiments, the soluble AXL variant polypeptide comprises at least one amino acid modification selected from the group consisting of 1) A19T, 2) T23M, 3) E26G, 4) E27G or E27K 5) G32S, 6) N33S, 7) 1381, 8) T44A, 9) H61Y, 10) D65N, 11) A72V, 12) S74N, 13) Q78E, 14) V79M, 15) Q86R, 16) D87G, 17) D88N, 18) 190M or 190V, 19) V92A, V92G or V92D, 20) 197R, 21) T98A or T98P, 22) T105M, 23) Q109R, 24) V112A, 25) F113L, 26) H116R, 27) T118A, 28) G127R or G127E, and 29) G129E and a combination thereof.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; and (d) glycine 127.

In some embodiments, the AXL variant polypeptide comprises amino acid changes relative to the wild-type AXL sequence (SEQ ID NO: 1) at the following positions: (a) aspartic acid 87 and (b) valine 92.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; (d) glycine 127 and (e) alanine 72.

In some embodiments, the AXL variant polypeptide comprises amino acid changes relative to the wild-type AXL sequence (SEQ ID NO: 1) at the following position: alanine 72.

In some embodiments, the AXL variant polypeptide glycine 32 residue is replaced with a serine residue, aspartic acid 87 residue is replaced with a glycine residue, valine 92 residue is replaced with an alanine residue, or glycine 127 residue is replaced with an arginine residue or a combination thereof.

In some embodiments, the AXL variant polypeptide residue aspartic acid 87 residue is replaced with a glycine residue or valine 92 residue is replaced with an alanine residue or a combination thereof.

In some embodiments, the AXL variant polypeptide alanine 72 residue is replaced with a valine residue.

In some embodiments, the AXL variant polypeptide glycine 32 residue is replaced with a serine residue, aspartic acid 87 residue is replaced with a glycine residue, valine 92 residue is replaced with an alanine residue, glycine 127 residue is replaced with an arginine residue or an alanine 72 residue is replaced with a valine residue or a combination thereof.

In some embodiments, the AXL variant comprises amino acid changes relative to the wild-type AXL sequence (SEQ ID NO: 1) at the following positions: (a) glutamic acid 26; (b) valine 79; (c) valine 92; and (d) glycine 127.

In some embodiments, the AXL variant polypeptide glutamic acid 26 residue is replaced with a glycine residue, valine 79 residue is replaced with a methionine residue, valine 92 residue is replaced with an alanine residue, or glycine 127 residue is replaced with an arginine residue or a combination thereof.

In some embodiments, the AXL variant polypeptide comprises at least an amino acid region selected from the group consisting of amino acid region 19-437, 130-437, 19-132, 21-121, 26-132, 26-121 and 1-437 of the wild-type AXL polypeptide (SEQ ID NO: 1), and wherein one or more amino acid modifications occur in said amino acid region.

In some embodiments, the AXL variant polypeptide comprises amino acid changes relative to the wild-type AXL sequence (SEQ ID NO: 1) at the following positions: (a) glycine 32; (b) aspartic acid 87; (c) alanine 72; and valine 92.

In some embodiments, the AXL variant polypeptide glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL variant is a fusion protein comprising an Fc domain, lacks a functional FN domain, and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72 and (d) valine 92.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, and valine 92 is replaced with an alanine residue or a combination thereof.

In some embodiments, the soluble AXL polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide has an affinity of at least about 1×10⁻⁹ M, 1×10⁻⁹ M, 1×10⁻¹⁹ M, 1×10⁻¹¹ M or 1×10⁻¹² M for GAS6.

In some embodiments, the soluble AXL variant polypeptide exhibits an affinity to GAS6 that is at least about 5-fold stronger, at least about 10-fold stronger or at least about 20-fold stronger than the affinity of the wild-type AXL polypeptide.

In some embodiments, the soluble AXL variant polypeptide further comprises a linker. In some embodiments, the linker comprises one or more (GLY)4SER units. In some embodiments, the linker comprises 1, 2, 3 or 5 (GLY)4SER units.

In some embodiments, the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, and of about 20.0 mg/kg. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 20 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

### BRIEF DESCRIPTION OF THE DRAWINGS

FIG. 1. Inhibition of GAS6-induced invasion/migration using an AXL decoy receptor. (A) soluble AXL decoy receptor (AVB-S6-500) and MDA-MB-231 Axl⁺ TNBC cells in serum free media were seeded in the top of a Matrigel-coated Boyden Chamber. Media with serum as a chemo-attractant was added to the chamber bottom. After 24 hours incubation, the number of cells that migrated across the Matrigel were counted and expressed as the fraction of invasive cells relative to a PBS control. (B) AVB-S6-500, OVCAR8 Axl+ovarian cancer cells, Type 1 collagen, 50 ng/mL GAS6, and growth media were seeded into microwells and incubated. On Day 6, the number of cells exhibiting the invasive phenotype were counted and expressed as the fraction of invasive cells relative to a PBS control. AVB-S6-500 over the range 1 to 100 pg/mL significantly inhibited GAS6-induced cell invasion/migration.

FIG. 2. Representative images from the AVB-S6-500 MDA-MB-231 cell invasion assay.

FIG. 3. Reduction of metastatic tumor burden using an AXL decoy receptor. Mice were inoculated intraperitoneally (IP) with SKOV3.ip ovarian cancer tumor cells (1×10⁶) and randomized into groups and AVB-S6-500 was administrated at 5, 10, or 20 mg/kg every other day (Q2D). Metastatic tumor burden was assessed after 24 days of dosing by counting all visible metastatic lesions in the peritoneal cavity and excising and weighing all diseased tissue to determine the overall weight (A) and number of metastases (B). AVB-S6-500 significantly reduced metastatic tumor burden when administered at 10 and 20 mg/kg.

FIG. 4. Superior efficacy using combination of an AXL decoy receptor and doxorubicin. Mice were inoculated intraperitoneally (IP) with SKOV3.ip ovarian cancer tumor cells (1×10⁶) and randomized into groups and AVB-S6-500 was administered at 20 mg/kg Q2D alone or combined twice per week 2 mg/kg doxorubicin (DOX). Metastatic tumor burden was assessed after 24 days of dosing. Comparison of the overall weight (A) and number (B) of metastases showed a significant benefit to combination therapy. AVB-S6-500 and doxorubicin combined significantly decreased mean weights of diseased tissue and cured 2 animals.

FIG. 5. Abrogation of serum GAS6 for ˜1 week in cynomolgus monkeys following a single dose at 5 mg/kg (1.7 mg/kg human equivalent dose) of an AXL decoy receptor. In cynomolgus monkeys, 5 mg/kg AVB-S6-500 resulted in abrogation of serum GAS6 for at least 168 hours and a NOAEL≥150 mg/kg/day was established in weekly repeat dosing studies.

FIG. 6. Abrogation of serum GAS6 for ˜1 week in human subjects following a single dose at 1 mg/kg (A) and 2.5 mg/kg (B) of an AXL decoy receptor.

FIG. 7. Abrogation of serum GAS6 for ˜1 week in human subjects following a single dose at 5 mg/kg (A) and 10 mg/kg (B) of an AXL decoy receptor.

FIG. 8. Line graph depicting mean (+/−) concentrations of AVB-S6-500 following a single IV Infusion of AVB-S6-500 in healthy subjects (N=6/dose).

FIG. 9. Line graph depicting mean (+/−) concentrations of serum GAS6 following a single iv infusion of AVB-S6-500 in healthy subjects (n=6/dose).

### MODE(S) FOR CARRYING OUT THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The term “tumor,” as used herein, refers to all neoplastic cell growth and proliferation, whether malignant or benign, and all pre-cancerous and cancerous cells and tissues.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, the cells of interest for detection, analysis, classification, or treatment in the present application include precancerous (e.g., benign), malignant, pre-metastatic, and non-metastatic cells.

The term “primary tumor” refers to all neoplastic cell growth and proliferation, whether malignant or benign, and all pre-cancerous and cancerous cells and tissues located at the anatomical site where the autonomous, unregulated growth of the cells initiated, for example the organ of the original cancerous tumor. Primary tumors do not include metastases.

The “pathology” of cancer includes all phenomena that compromise the well-being of the patient. This includes, without limitation, abnormal or uncontrollable cell growth, primary tumor growth and formation, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor (e.g., the organ containing the primary tumor). Metastasis can also be defined as several steps of a process, such as the departure of cancer cells from an original tumor site (e.g., primary tumor site) and migration and/or invasion of cancer cells to other parts of the body.

Depending on the nature of the cancer, an appropriate patient sample is obtained. As used herein, the phrase “cancerous tissue sample” refers to any cells obtained from a cancerous tumor. In the case of solid tumors which have not metastasized (for example a primary tumor), a tissue sample from the surgically removed tumor will typically be obtained and prepared for testing by conventional techniques.

The definition of an appropriate patient sample encompasses blood and other liquid samples of biological origin, solid tissue samples such as a biopsy specimen or tissue cultures or cells derived there from and the progeny thereof. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

Tumors of interest for treatment with the methods of the invention include solid tumors, e.g. carcinomas, gliomas, melanomas, sarcomas, and the like. Ovarian cancer and breast cancer is of particular interest. Carcinomas include a variety of adenocarcinomas, for example in prostate, lung, etc.; adernocartical carcinoma; hepatocellular carcinoma; renal cell carcinoma, ovarian carcinoma, carcinoma in situ, ductal carcinoma, carcinoma of the breast, basal cell carcinoma; squamous cell carcinoma; transitional cell carcinoma; colon carcinoma; nasopharyngeal carcinoma; multilocular cystic renal cell carcinoma; oat cell carcinoma, large cell lung carcinoma; small cell lung carcinoma; etc. Carcinomas may be found in prostrate, pancreas, colon, brain (e.g., glioblastoma), lung, breast, skin, etc. Including in the designation of soft tissue tumors are neoplasias derived from fibroblasts, myofibroblasts, histiocytes, vascular cells/endothelial cells and nerve sheath cells. Tumors of connective tissue include sarcomas; histiocytomas; fibromas; skeletal chondrosarcoma; extraskeletal myxoid chondrosarcoma; clear cell sarcoma; fibrosarcomas, etc. Hematologic cancers include leukemias and lymphomas, e.g. cutaneous T cell lymphoma, acute myeloid leukemia (AML), chronic myeloid leukemia (CML), acute lymphoblastic leukemia (ALL), non-Hodgkins lymphoma (NHL), etc. In some embodiments, the cancer is ovarian cancer. In some embodiments, the cancer is a cancer that overexpresses the biomarker GAS6 and/or AXL. In some embodiments, the patient previously responded to treatment with an anti-cancer therapy, but, upon cessation of therapy, suffered relapse (hereinafter “a recurrent cancer”). In some embodiments, the cancer is resistant to standard therapies. In some embodiments, the cancer is a chemoresistant cancer. In some embodiments, the cancer is a platinum resistant cancer.

“In combination with”, “combination therapy” and “combination products” refer, in certain embodiments, to the concurrent administration to a patient of a first therapeutic and the compounds as used herein. In some embodiments, the combination products are administered non-concurrently. When administered in combination, each component can be administered at the same time or sequentially in any order at different points in time. Thus, each component can be administered separately but sufficiently closely in time so as to provide the desired therapeutic effect.

As used herein, the phrase “disease-free survival,” refers to the lack of such tumor recurrence and/or invasion and the fate of a patient after diagnosis, with respect to the effects of the cancer on the life-span of the patient. The phrase “overall survival” refers to the fate of the patient after diagnosis, despite the possibility that the cause of death in a patient is not directly due to the effects of the cancer. The phrases, “likelihood of disease-free survival”, “risk of recurrence” and variants thereof, refer to the probability of tumor recurrence or spread in a patient subsequent to diagnosis of cancer, wherein the probability is determined according to the process of the invention.

The compounds having the desired pharmacological activity may be administered in a physiologically acceptable carrier to a host to modulate AXL/GAS6 function. The therapeutic agents may be administered in a variety of ways, orally, topically, parenterally e.g. intravenous, subcutaneously, intraperitoneally, by viral infection, intravascularly, etc. Intravenous delivery is of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

“Inhibitors,” “activators,” and “modulators” of AXL or its ligand GAS6 are used to refer to inhibitory, activating, or modulating molecules, respectively, identified using in vitro and in vivo assays for receptor or ligand binding or signaling, e.g., ligands, receptors, agonists, antagonists, and their homologs and mimetics.

The terms “polypeptide,” “peptide” and “protein” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms “antibody” and “antibodies” are used interchangeably herein and refer to a polypeptide capable of interacting with and/or binding to another molecule, often referred to as an antigen. Antibodies can include, for example “antigen-binding polypeptides” or “target-molecule binding polypeptides.” Antigens of the present invention can include for example any polypeptides described in the present invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an a-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. All single letters used in the present invention to represent amino acids are used according to recognized amino acid symbols routinely used in the field, e.g., A means Alanine, C means Cysteine, etc. An amino acid is represented by a single letter before and after the relevant position to reflect the change from original amino acid (before the position) to changed amino acid (after position). For example, A19T means that amino acid alanine at position 19 is changed to threonine.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer, including without limitation, adenocarcinoma of the ovary or prostate, breast cancer, glioblastoma, etc., including those who have undergone or are candidates for resection (surgery) to remove cancerous tissue. Subjects may be human, but also include other mammals, particularly those mammals useful as laboratory models for human disease, e.g. mouse, rat, etc.

The definition of an appropriate patient sample encompasses blood and other liquid samples of biological origin, solid tissue samples such as a biopsy specimen or tissue cultures or cells derived there from and the progeny thereof. The definition also includes samples that have been manipulated in any way after their procurement, such as by treatment with reagents; washed; or enrichment for certain cell populations, such as endometrial cells, kidney disease cells, inflammatory disease cells and/or transplant rejection (GVHD) cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample comprising polynucleotides and/or polypeptides that is obtained from a patient's sample cell (e.g., a cell lysate or other cell extract comprising polynucleotides and/or polypeptides); and a sample comprising sample cells from a patient. A biological sample comprising a sample cell from a patient can also include normal, non-diseased cells.

The term “diagnosis” is used herein to refer to the identification of a molecular or pathological state, disease or condition, such as the identification of a virus infection.

As used herein, the terms “treatment,” “treating,” and the like, refer to administering an agent, or carrying out a procedure for the purposes of obtaining an effect. The effect may be prophylactic in terms of completely or partially preventing a disease or symptom thereof and/or may be therapeutic in terms of effecting a partial or complete cure for a disease, and/or symptoms of the disease. “Treatment,” as used herein, covers any treatment of any virus infection or exposure in a mammal, particularly in a human, and includes: (a) preventing the infection; (b) inhibiting the infection, i.e., arresting its development; and (c) relieving the disease, i.e., causing regression of infection.

Treating may refer to any indicia of success in the treatment or amelioration or prevention of cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions. The term “therapeutic effect” refers to the reduction, elimination, or prevention of the disease, symptoms of the disease, or side effects of the disease in the subject.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient “means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” refers to the amount of a compound that, when administered to a subject for treating breast or ovarian cancer, is sufficient to affect such treatment of the cancer. The “therapeutically effective amount” may vary depending, for example, on the soluble AXL variant polypeptide selected, the stage of the cancer, the age, weight and/or health of the patient and the judgment of the prescribing physician. An appropriate amount in any given instance may be readily ascertained by those skilled in the art or capable of determination by routine experimentation.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).).

As used herein, the term “progression free survival” means the time period for which a subject having a disease (e.g. cancer) survives, without a significant worsening of the disease state. Progression free survival may be assessed as a period of time in which there is no progression of tumor growth and/or wherein the disease status of a patient is not determined to be a progressive disease. In some embodiments, progression free survival of a subject having cancer is assessed by evaluating tumor (lesion) size, tumor (lesion) number, and/or metastasis.

As used herein, “objective response rate (”ORR″) is defined as the proportion of patients with tumor size reduction of a predefined amount and for a minimum period of time. Response duration is usually measured from the time of initial response until documented tumor progression. Generally, the ORR can be defined as the sum of partial responses plus complete responses.

“Concomitant administration” of a known cancer therapeutic drug with a pharmaceutical composition of the present invention means administration of the drug and AXL variant at such time that both the known drug and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

AXL, MER, Tyro3 and GAS6, as well as related pathways, have been described in WO2011/091305, as well as U.S. application Ser. Nos. 13/554,954 and 13/595,936; all of which are incorporated herein by reference in their entireties for all purposes.

### Exemplary Embodiments

Methods of the present invention include treating, reducing, or preventing metastasis of cancers, by administering a soluble AXL variant polypeptide as described herein. In one aspect, the present invention provides methods for the treatment of human metastatic cancers, comprising the administration of a soluble AXL polypeptide that lacks the AXL transmembrane domain and has at least one mutation relative to wild-type AXL that increases affinity of the AXL polypeptide binding to GAS6 compared to wild-type AXL.

In some embodiments, the methods prolong progression free survival as compared to control. In some embodiments, the methods prolong overall survival as compared to control. In some embodiments, the methods achieve improved progression free survival as compared to control. In some embodiments, the methods achieve improved chemotherapy free interval as compared to control. In some embodiments, the methods achieve improved time to first subsequent therapy as compared to control. In some embodiments, the methods achieve improved time to second subsequent therapy as compared to control. In some embodiments, the methods have been determined to not have a detrimental effect on Quality of Life as determined by FOSI and/or EQ-5D-5L.

Cancers of interest include solid tumors and hematologic malignancies. In various embodiments, the cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

Ovarian cancer is the 5th overall cause for cancer death in women and represents 5% of all cancer deaths in women. In 2014 it is estimated that there will be 21,980 new cases of ovarian cancer and an estimated 14,270 women will die of this disease. The expected incidence of epithelial ovarian cancer in women in the United States in 2012 is approximately 22,280 (15,500 deaths) and in Europe in 2012 was estimated at 65,538 patient cases (42,704 deaths). High-grade serous ovarian cancer is the most common subtype and displays widespread genomic instability, indicating likely a defect in homologous recombination (Bowtell D D, Nat Rev Cancer 2010; 10: 803-8). At diagnosis, most women present with advanced disease, which accounts for the high mortality rate. Initial chemotherapy consists of either taxane or platinum chemotherapy or a combination of both. While approximately 75% of patients respond to front line therapy 70% of those eventually relapse within 1 to 3 years. There is a significant unmet need due to the high recurrence rate, despite an initially high response rate. Attempts to improve the standard two-drug chemotherapy (carboplatin and paclitaxel) by adding a third cytotoxic drug (topotecan, gemcitabine, or doxil) have failed (du Bois et al, 2006 and Pfisterer et al, 2006). Maintenance therapy after the achievement of a response from initial chemotherapy may represent an approach to provide clinical benefit by delaying disease progression side effects, delaying the need for toxic chemotherapy and prolonging overall survival. However, there is currently no widely accepted standard of care in the ovarian cancer maintenance setting.

In some embodiments, the cancer is ovarian cancer. In some embodiments, the ovarian cancer is resistant to standard therapies. In some embodiments, the recurrent and/or platinum resistant cancer is ovarian cancer. In some embodiments, the ovarian cancer is platinum resistant ovarian cancer at the commencement of soluble AXL variant polypeptide therapy. In some embodiments, the ovarian cancer is recurrent, platinum resistant ovarian cancer at the commencement of soluble AXL variant polypeptide therapy. In some embodiments, the ovarian cancer responded to the most recent platinum-based chemotherapy regimen prior to commencement of soluble AXL variant polypeptide therapy. In some embodiments, response to the most recent platinum-based chemotherapy regimen is a complete response. In some embodiments, response to the most recent platinum-based chemotherapy regimen is a partial response. In some embodiments, the ovarian cancer responded to the penultimate platinum-based chemotherapy regimen prior to commencement of soluble AXL variant polypeptide therapy.

In another aspect, the present invention provides methods for the treatment of cancer, comprising the administration of a soluble AXL polypeptide that lacks the AXL transmembrane domain and has at least one mutation relative to wild-type AXL that increases affinity of the AXL polypeptide binding to GAS6 compared to wild-type AXL, in combination with a second therapy selected from the group consisting of: surgery, cytoreductive therapy, cytotoxic chemotherapy, and immunotherapy. In some embodiments, the combination may be synergistic.

In some embodiments, the combination therapy comprises anti-proliferative, or cytoreductive therapy. Anti-proliferative, or cytoreductive therapy is used therapeutically to eliminate tumor cells and other undesirable cells in a host and includes the use of therapies such as delivery of ionizing radiation, and administration of chemotherapeutic agents. For example, ionizing radiation (IR) is used to treat about 60% of cancer patients, by depositing energy that injures or destroys cells in the area being treated, and for the purposes of the present invention may be delivered at conventional doses and regimens, or at reduced doses. Radiation injury to cells is nonspecific, with complex effects on DNA. The efficacy of therapy depends on cellular injury to cancer cells being greater than to normal cells. Radiotherapy may be used to treat every type of cancer. Some types of radiation therapy involve photons, such as X-rays or gamma rays. Another technique for delivering radiation to cancer cells is internal radiotherapy, which places radioactive implants directly in a tumor or body cavity so that the radiation dose is concentrated in a small area. A suitable dose of ionizing radiation may range from at least about 2 Gy to not more than about 10 Gy, usually about 5 Gy. A suitable dose of ultraviolet radiation may range from at least about 5 J/m² to not more than about 50 J/m², usually about 10 J/m². The sample may be collected from at least about 4 and not more than about 72 hours following ultraviolet radiation, usually around about 4 hours.

Chemotherapeutic agents are well-known in the art and are used at conventional doses and regimens, or at reduced dosages or regimens, including for example, topoisomerase inhibitors such as anthracyclines, including the compounds daunorubicin, adriamycin (doxorubicin), epirubicin, idarubicin, anamycin, MEN 10755, and the like. Other topoisomerase inhibitors include the podophyllotoxin analogues etoposide and teniposide, and the anthracenediones, mitoxantrone and amsacrine. Other anti-proliferative agent interferes with microtubule assembly, e.g. the family of vinca alkaloids. Examples of vinca alkaloids include vinblastine, vincristine; vinorelbine (NAVELBINE); vindesine; vindoline; vincamine; etc. DNA-damaging agent include nucleotide analogs, alkylating agents, etc. Alkylating agents include nitrogen mustards, e.g. mechlorethamine, cyclophosphamide, melphalan (L-sarcolysin), etc.; and nitrosoureas, e.g. carmustine (BCNU), lomustine (CCNU), semustine (methyl-CCNU), streptozocin, chlorozotocin, etc. Nucleotide analogs include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc. Other chemotherapeutic agents of interest include metal complexes, e.g. cisplatin (cis-DDP), carboplatin, oxaliplatin, etc.; ureas, e.g. hydroxyurea; gemcitabine, and hydrazines, e.g. N-methylhydrazine. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

In some embodiments, the combination therapy will comprise immunotherapy. As used herein, the term “immunotherapy” refers to cancer treatments which include, but are not limited to treatment using depleting antibodies to specific tumor antigens (see, e.g., reviews by Blattman and Greenberg, Science, 305:200, 2004; Adams and Weiner, Nat Biotech, 23:1147, 2005; Vogal et al. J Clin Oncology, 20:719, 2002; Colombat et al., Blood, 97:101, 2001); treatment using antibody-drug conjugates (see, e.g., Ducry, Laurent (Ed.) Antibody Drug Conjugates. In: Methods in Molecular Biology. Book 1045. New York (N.Y.), Humana Press, 2013; Nature Reviews Drug Discovery 12, 259-260, April 2013); treatment using agonistic, antagonistic, or blocking antibodies to co-stimulatory or co-inhibitory molecules (immune checkpoints) such as CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, Oct 2014), OX-40, CD137, GITR, LAGS, TIM-3, and VISTA (see, e.g., Sharon et al., Chin J Cancer., 33(9): 434-444, Sep 2014; Hodi et al., N Engl J Med, 2010; Topalian et al., N Engl J Med, 366:2443-54, 2012); treatment using bispecific T cell engaging antibodies (BITE®) such as blinatumomab (see, e.g., U.S. Pat. No. 9,260,522; US Patent Application No. 20140302037); treatment involving administration of biological response modifiers such as IL-2, IL-12, IL-15, IL-21, GM-CSF, IFN-α, IFN-β, and IFN-γ□□see, e.g., Sutlu T et al.,, Journ of Internal Medicine, 266(2):154-181, 2009; Joshi S PNAS USA, 106(29):12097-12102, 2009; Li Y et al., Journal of Translational Medicine, 7:11, 2009); treatment using therapeutic vaccines such as sipuleucel-T (see, e.g., Kantoff PW New England Journal of Medicine, 363(5):411-422, 2010; Schlom J., Journal of the National Cancer Institutes, 104(8):599-613, 2012); treatment using dendritic cell vaccines, or tumor antigen peptide vaccines; treatment using chimeric antigen receptor (CAR)-T cells (see, e.g., Rosenberg SA Nature Reviews Cancer, 8(4):299-308, 2008; Porter DL et al, New England Journal of Medicine, 365(8):725-733, 2011; Grupp SA et al., New England Journal of Medicine, 368(16):1509-151, 2013; U.S. Pat. Nos. 9,102,761; 9,101,584); treatment using CAR-NK cells (see, e.g., Glienke et al., Front Pharmacol, 6(21):1-7, Feb 2015); treatment using tumor infiltrating lymphocytes (TILs)(see e.g., Wu et al, Cancer J., 18(2): 160-175, 2012); treatment using adoptively transferred anti-tumor T cells (ex vivo expanded and/or TCR transgenic)(see e.g., Wrzesinski et al., J Immunother, 33(1): 1-7, 2010); treatment using TALL-104 cells; and treatment using immunostimulatory agents such as Toll-like receptor (TLR) agonists CpG and imiquimod (see, e.g., Krieg, Oncogene, 27:161-167, 2008; Lu, Front Immunol, 5(83):1-4, March 2014).

Immunotherapy focused on utilization of depleting antibodies to specific tumor antigens have been explored with much success (see, e.g., reviews by Blattman and Greenberg, Science, 305:200, 2004; Adams and Weiner, Nat Biotech, 23:1147, 2005). A few examples of such tumor antigen-specific, depleting antibodies are HERCEPTIN® (anti-Her2/neu mAb)(Baselga et al., J Clin Oncology, Vol 14:737, 1996; Baselga et al., Cancer Research, 58:2825, 1998; Shak, Semin. Oncology, 26 (Supp112):71, 1999; Vogal et al. J Clin Oncology, 20:719, 2002); and RITUXAN® (anti-CD20 mAb)(Colombat et al., Blood, 97:101, 2001). Unfortunately, while clearly having made a mark in oncology treatment, as monotherapy they generally work in only about 30% of the individuals and with a partial response. Moreover, many individuals eventually become refractory or relapse after treatment with these antibody-containing regimens.

Treatment using agonistic, antagonistic, or blocking antibodies to co-stimulatory or co-inhibitory molecules (immune checkpoints) has been an area of extensive research and clinical evaluation. Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll DM., Nat Rev Cancer, 12:252-64, 2012). Accordingly, treatment utilizing antibodies to immune checkpoint molecules including, e.g., CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, Oct 2014), and OX-40, CD137, GITR, LAGS, TIM-3, and VISTA (see, e.g., Sharon et al., Chin J Cancer., 33(9): 434-444, Sep 2014; Hodi et al., N Engl J Med, 2010; Topalian et al., N Engl J Med, 366:2443-54) are being evaluated as new, alternative immunotherapies to treat patients with proliferative diseases such as cancer, and in particular, patients with refractory and/or recurrent cancers.

Treatment using chimeric antigen receptor (CAR) T cell therapy is an immunotherapy in which the patient's own T cells are isolated in the laboratory, redirected with a synthetic receptor to recognize a particular antigen or protein, and reinfused into the patient. CARs are synthetic molecules that minimally contain: (1) an antigen-binding region, typically derived from an antibody, (2) a transmembrane domain to anchor the CAR into the T cells, and (3) 1 or more intracellular T cell signaling domains. A CAR redirects T cell specificity to an antigen in a human leukocyte antigen (HLA)-independent fashion, and overcomes issues related to T cell tolerance (Kalos M and June CH, Immunity, 39(1):49-60, 2013). Over the last 5 years, at least 15 clinical trials of CAR-T cell therapy have been published. A new wave of excitement surrounding CAR-T cell therapy began in August 2011, when investigators from the University of Pennsylvania (Penn) published a report on 3 patients with refractory chronic lymphocytic leukemia (CLL) who had long-lasting remissions after a single dose of CART cells directed to CD 19 (Porter DL, et al., N Engl J Med., 365(8):725-733, 2011).

In contrast to donor T cells, natural killer (NK) cells are known to mediate anti-cancer effects without the risk of inducing graft-versus-host disease (GvHD). Accordingly, alloreactive NK cells are now also the focus of considerable interest as suitable and powerful effector cells for cellular therapy of cancer. Several human NK cell lines have been established, e.g., NK-92, HANK-1, KHYG-1, NK-YS, NKG, YT, YTS, NKL and NK3.3 (Kornbluth,J., et al., J. Immunol. 134, 728-735, 1985; Cheng,M. et al., Front.Med. 6:56, 2012) and various CAR expressing NK cells (CAR-NK) have been generated. Immunotherapy using CAR expressing NK cells (CAR-NK) is an active area of research and clinical evaluation (see, e.g., Glienke et al., Front Pharmacol, 6(21):1-7, Feb. 2015).

Bispecific T-cell engager molecules (BiTE®s) constitute a class of bispecific single-chain antibodies for the polyclonal activation and redirection of cytotoxic T cells against pathogenic target cells. BiTE®s are bispecific for a surface target antigen on cancer cells, and for CD3 on T cells. BiTE®s are capable of connecting any kind of cytotoxic T cell to a cancer cell, independently of T-cell receptor specificity, costimulation, or peptide antigen presentation. a unique set of properties that have not yet been reported for any other kind of bispecific antibody construct, namely extraordinary potency and efficacy against target cells at low T-cell numbers without the need for T-cell co-stimulation (Baeuerle et al., Cancer Res, 69(12):4941-4, 2009). BiTE antibodies have so far been constructed to more than 10 different target antigens, including CD19, EpCAM, Her2/neu, EGFR, CD66e (or CEA, CEACAMS), CD33, EphA2, and MCSP (or HMW-MAA)(Id.) Treatment using BiTE® antibodies such as blinatumomab (Nagorsen, D. et al., Leukemia & Lymphoma 50(6): 886-891, 2009) and solitomab (Amann et al., Journal of Immunotherapy 32(5): 452-464, 2009) are being clinically evaluated.

In some embodiments, the second therapy will comprise administration of a PARP inhibitor. Poly(ADP-ribose) polymerases (PARPs) are a family of enzymes involved in various activities in response to DNA damage. PARP-1 is a key DNA repair enzyme that mediates single strand break (SSB) repair through the base excision repair (BER) pathway. PARP inhibitors have been demonstrated to selectively kill tumor cells that harbor BRCA1 and BRCA2 mutations. In addition, pre-clinical and preliminary clinical data suggest that PARP inhibitors are selectively cytotoxic for tumors with homologous recombination repair deficiency caused by dysfunction of genes other than BRCA1 or BRCA2. In some embodiments, the PARP inhibitor is selected from the group consisting of ABT-767, AZD 2461, BGB-290, BGP 15, CEP 9722, E7016, E7449, fluzoparib, IN01001, JPI 289, MP 124, niraparib, olaparib, 0N02231, rucaparib, SC 101914, talazoparib, veliparib, WW 46, or salts or derivatives thereof. In some embodiments, the anti-PARP therapy is administered at a dose equivalent to about 100 mg, about 200 mg, or about 300 mg of niraparib or a salt or derivative thereof. In some embodiments, the anti-PARP therapy is administered at a dose equivalent to about 100 mg of niraparib or a salt or derivative thereof. In some embodiments, the anti-PARP therapy is administered at a dose equivalent to about 200 mg of niraparib or a salt or derivative thereof. In certain embodiments, the anti-PARP therapy is administered at a dose equivalent to about 300 mg of niraparib or a salt or derivative thereof.

The AXL variant may be administered prior to, concurrently with, or following the second therapy, usually within at least about 1 week, at least about 5 days, at least about 3 days, at least about 1 day. The AXL variant may be delivered in a single dose, or may be fractionated into multiple doses, e.g. delivered over a period of time, including daily, bidaily, semi-weekly, weekly, etc. The effective dose will vary with the route of administration, the specific agent, the dose of cytoreductive agent, and the like, and may be determined empirically by one of skill in the art. A useful range for i.v. administered polypeptides may be empirically determined, for example at least about 0.1 mg/kg body weight; at least about 0.5 mg/kg body weight; at least about 1 mg/kg body weight; at least about 2.5 mg/kg body weight; at least about 5 mg/kg body weight; at least about 10 mg/kg body weight; at least about 20 mg/kg body weight; or more. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 20 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

In still some embodiments, therapeutic entities of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

In yet other embodiments, methods of the present invention include administering to a subject in need of treatment a therapeutically effective amount or an effective dose of a therapeutic entity (e.g., inhibitor agent) of the present invention. In some embodiments, effective doses of the therapeutic entity of the present invention, e.g. for the treatment of primary or metastatic cancer, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

In some embodiments, the dosage may range from about 0.0001 to 100 mg/kg, and more usually 0.01 to 5 mg/kg, of the host body weight. For example, dosages can be 1 mg/kg body weight or 10 mg/kg body weight or within the range of 1-10 mg/kg. In some embodiments, the dosage of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, and of about 20.0 mg/kg. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 20 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

In some embodiments, the treatment regime entails administration once per every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing primary tumor formation or tumor metastasis or tumor invasion of AML, ovarian cancer, breast cancer, lung cancer, liver cancer, colon cancer, gallbladder cancer, pancreatic cancer, prostate cancer, and/or glioblastoma.

In still yet some other embodiments, for prophylactic applications, pharmaceutical compositions or medicaments are administered to a patient susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In still yet some other embodiments, for therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if there is a recurrence of the cancer.

According to the present invention, compositions for the treatment of primary or metastatic cancer can be administered by parenteral, topical, intravenous, intratumoral, oral, subcutaneous, intraarterial, intracranial, intraperitoneal, intranasal or intramuscular means. The most typical route of administration is intravenous or intratumoral although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. I n general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. In some embodiments, the composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein. Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.Preferably, a therapeutically effective dose of the polypeptide compositions described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions of the invention and instructions for use. The kit can further contain a least one additional reagent, for example a cytoreductive drug. The compositions may be provided in a unit dose formulation. Kits typically include a label indicating the intended use of the contents of the kit. The term label includes any writing, or recorded material supplied on or with the kit, or which otherwise accompanies the kit.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize or be able to ascertain using no more than routine experimentation, many equivalents to the specific embodiments of the invention described herein. Such equivalents are intended to be encompassed by the appended claims.

### EXPERIMENTAL

### Example 1

### Inhibition of GAS6-Induced Invasion/Migration using an AXL Decoy Receptor

Inhibition of GAS6-induced invasion/migration was evaluated in models for triple-negative breast (MDA-MB-231) and ovarian (OVCAR8) cancers using the Corning® Matrigel® or collagen invasion assays, respectively, and using an AXL decoy receptor which comprises a soluble AXL variant polypeptide comprising amino acid changes relative to wild-type AXL sequence (SEQ ID NO:1) at the following positions: (a) glycine 32; (b) aspartic acid 87; (c) alanine 72; (d) valine 92; and (e) glycine 127, lacking the AXL transmembrane domain, lacking a functional FN domain, and comprising an Fc domain linked to the soluble AXL variant polypeptide by a peptide linker (hereinafter referred to as AVB-S6-500).

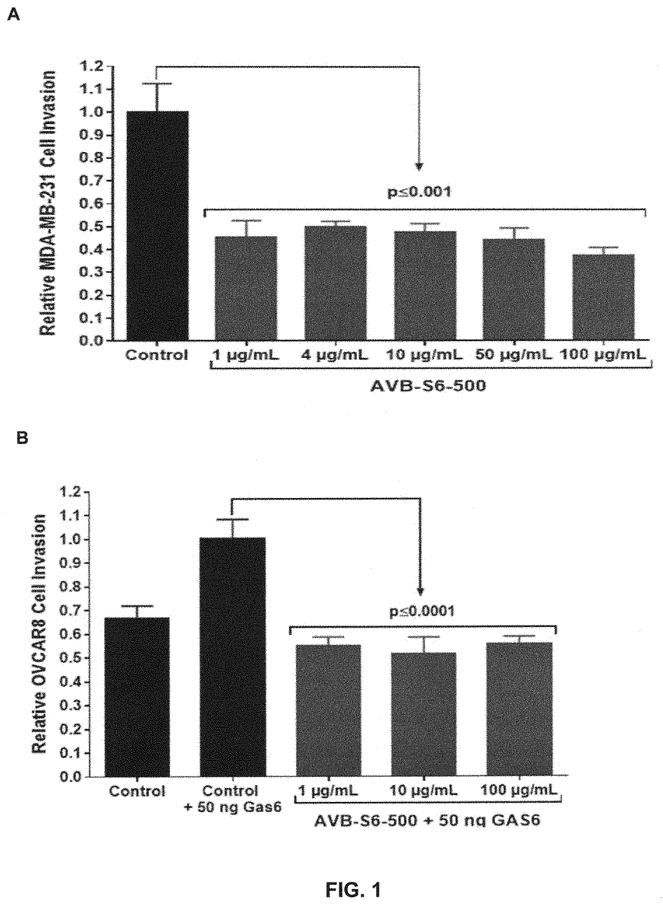

AVB-S6-500 and MDA-MB-231 Axl+ TNBC cells in serum free media were seeded in the top of a Matrigel-coated Boyden Chamber. Media with serum as a chemo-attractant was added to the chamber bottom. After 24 hours incubation, the number of cells that migrated across the Matrigel were counted and expressed as the fraction of invasive cells relative to a PBS control (FIG. 1A). AVB-S6-500, OVCAR8 Axl+ovarian cancer cells, Type 1 collagen, 50 ng/mL GAS6, and growth media were seeded into microwells and incubated. On Day 6, the number of cells exhibiting the invasive phenotype were counted and expressed as the fraction of invasive cells relative to a PBS control (FIG. 1B). AVB-S6-500 over the range 1 to 100 pg/mL significantly inhibited GAS6-induced cell invasion/migration.

### Example 2

### Determination of IC₅₀ Values and Comparison of an AXL Decoy Receptor Versus a Tyrosine Kinase Inhibitor in an MDA-MB-231 Cell Invasion Assay

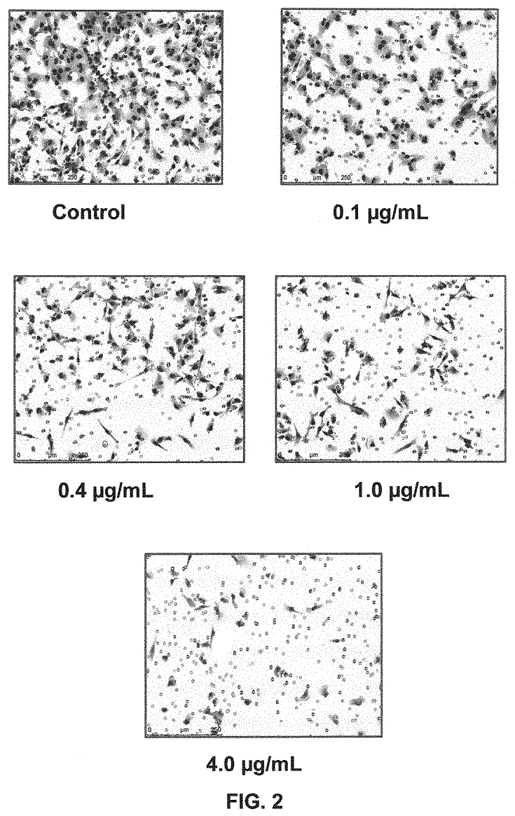

AVB-S6-500 IC₅₀ values were determined in a MDA-MB-231 cell invasion assay ±50 nM GAS6 and in a cell viability assay and compared to the IC₅₀ of an approved tyrosine kinase inhibitor, bosutinib. Representative images from the AVB-S6-500 treated MDA-MB-231 cells are depicted in FIG. 2. As shown in Table 1, AVB-S6-500 was ˜100-fold more potent than bosutinib in inhibiting cell invasion and did not affect cell viability for a panel of 8 diverse cancer cell lines (colon, breast, AML, ovarian, pancreatic, and NSCLC) compared to seven cytotoxic/chemotherapy standard of care (SOC) drugs.

TABLE 1
IC₅₀ [μM]

| Assay | AVB-S6-500 | AVB-S6-500 + GAS6 | Bosutinib | SOC (n −= 7) |
|---|---|---|---|---|
| MDA-MB-231 cell invasion | 6.5 × 10⁻⁴ | 3.9 × 10⁻⁴ | 2.4 × 10⁻² | — |
| Cancer cell viability (n = 8) | >100 | — | — | 0.8 ± 0.2 (n = 56) |

### Example 3

### Reduction of Metastatic Tumor Burden using an AXL Decoy Receptor

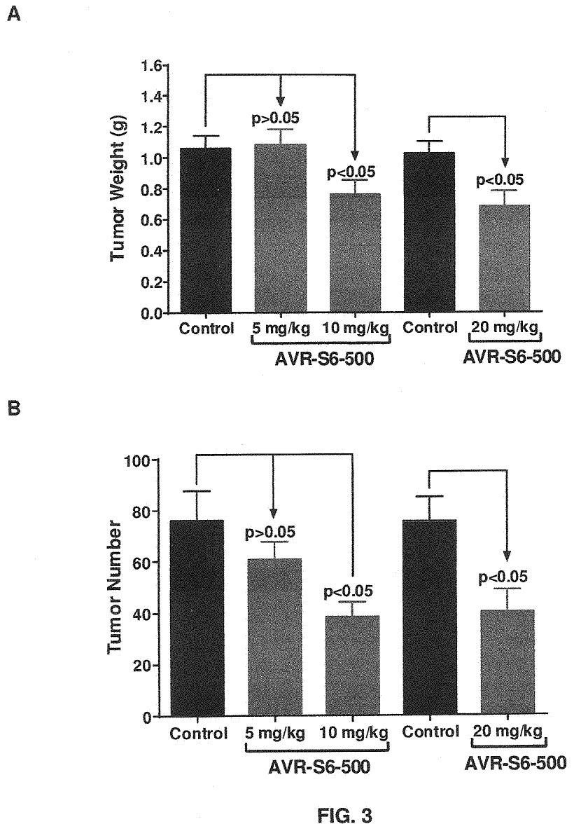

Mice were inoculated intraperitoneally (IP) with SKOV3.ip ovarian cancer tumor cells (1×10⁶) and randomized into groups and AVB-S6-500 was administrated at 5, 10, or 20 mg/kg every other day (Q2D). Metastatic tumor burden was assessed after 24 days of dosing by counting all visible metastatic lesions in the peritoneal cavity and excising and weighing all diseased tissue to determine the overall weight (FIG. 3A) and number of metastases (FIG. 3B). AVB-S6-500 significantly reduced metastatic tumor burden when administered at 10 and 20 mg/kg. AVB-S6-500 monotherapy in the SKOV3.IP mouse xenograft model significantly decreased mean number and weight of macroscopic metastatic lesions at 10 and 20 mg/kg Q2D (equivalent to 2.5-5 mg/kg/week in humans) and abrogated serum free GAS6 levels.

### Example 4

### Superior Efficacy using Combination of an AXL Decoy Receptor and Doxorubicin

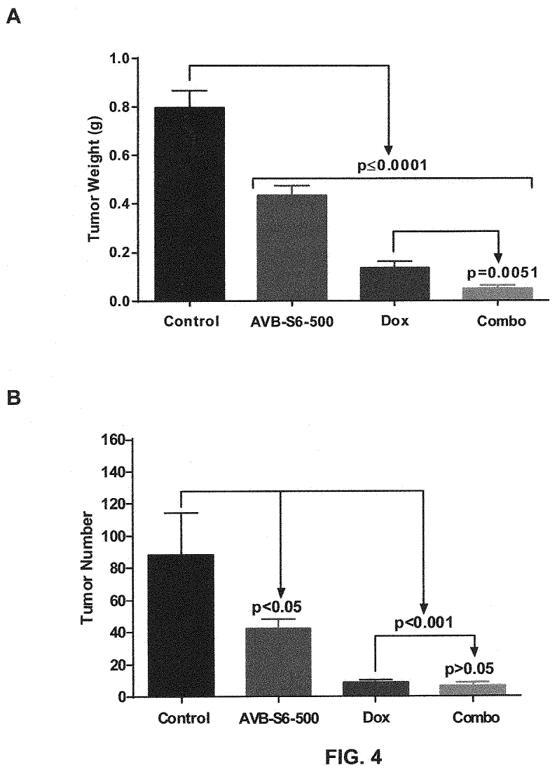

Mice were inoculated intraperitoneally (IP) with SKOV3.ip ovarian cancer tumor cells (1×10⁶) and randomized into groups and AVB-S6-500 was administered at 20 mg/kg Q2D alone or combined twice per week 2 mg/kg doxorubicin (DOX). Metastatic tumor burden was assessed after 24 days of dosing. Comparison of the overall weight (FIG. 4A) and number of metastases (FIG. 4B) showed a significant benefit to combination therapy. AVB-S6-500 and doxorubicin combined significantly decreased mean weights of diseased tissue and cured 2 animals.

### Example 5

### PK Studies

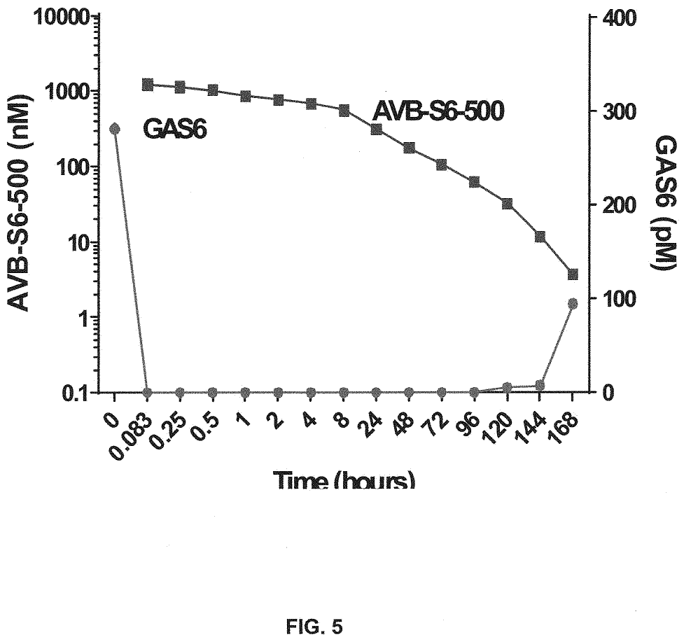

Mouse studies established a relationship between depletion of sGAS6 (drug target) and anti-metastatic effect. Doses that were effective in mouse studies were associated with abrogation of serum GAS6 levels in mice and equate to 0.5-1.7 mg/kg in humans. In PK studies in cynomolgus monkeys, AVB-S6-500 demonstrated abrogation of serum GAS6 for -1 week in cynomolgus monkeys following a single dose at 5 mg/kg (1.7 mg/kg human equivalent dose) (FIG. 5), a desirable profile for a weekly IV infusion in humans.

Due to the observed preclinical relationship between serum GAS6 (sGAS6) depletion and anti-metastatic activity, sGAS6 was identified a useful biomarker in an pharmacodynamic [PD] assay to assess sGAS6 levels in all nonclinical studies, including GLP toxicology. Very consistent PK/PD observed in the studies.

### Example 6

### Safety Studies

AVB-S6-500 administered to mice and monkeys was well-tolerated following single and repeat dosing at much higher doses than those needed for the desired biological effect. There were no treatment-related mortalities or adverse effects for single or twice weekly slow bolus intravenous injections (IV) at doses of 25, 50 and 100 mg/kg (50, 100 and 200 mg/kg/week) in male and female CD-1 mice or for four weekly 30 minute IV infusions at doses of 30, 100 and 150 mg/kg in monkeys. All doses provided complete abrogation of serum GAS6 levels for the entire study period. The no-observed-adverse-effect-levels (NOAELs) were at 200 mg/kg/week (top dose) in mice and 150 mg/kg/week in monkeys, respectively. Pharmacokinetic/pharmacodynamic modeling as well as extrapolation of mouse efficacious doses predicts 1.5-5 mg/kg AVB-S6-500 may be efficacious in humans. Very consistent PK/PD is observed in the nonclinical studies using sGAS6 as a biomarker.

AVB-S6-500 was shown to be effective in reducing metastatic cancer burden in human breast and ovarian cancer xenograft models and safe in cynomolgus monkeys and mice at much higher doses. These results are similar to those for predecessor decoy receptors with demonstrated efficacy and safety across many oncology models and support the safe use of AVB-S6-500 in healthy volunteers.

Modeling of animal PK/PD was used to guide dosing in human studies considering elevated sGAS6 seen in cancer patients. Specifically, the toxicology profile allowed dosing in healthy volunteers and GLP toxicology studies combined with PD guided dose selection for first in human study. The effect of GAS6 on the clearance of AVB-S6-500 was incorporated into a target-mediated drug disposition (TMDD) model, providing parallel linear and nonlinear clearance of AVB-S6. Simulations of human GAS6 suppression were performed for the dose levels of 1, 2.5, 5, and 10 mg/kg using monkey data. Considering potentially higher sGAS6 levels in cancer patients and dosing regimens of combination chemotherapies, different AVB-S6 dosing regimens were modeled to predict target coverage with doses to be used in the oncology studies. Using the target mediated drug disposition (TMDD) model, the human dose estimated to be efficacious ranged from 1.5 mg/kg (to ensure GAS6 levels remain at least 50% less than baseline) to 5 mg/kg (to ensure 97% abrogation of free GAS6 and allowing for a 3-fold increase in GAS6 levels relative to normal levels).

### Example 7

### Single-Blind, Randomized, Placebo-Controlled, Phase 1, Single Ascending-Dose and Repeat-Dose, Safety and Tolerability Study of Intravenous AVB-S6-500 in Healthy Subjects

The safety and tolerability of a single ascending dose (SAD) of intravenous AVB-S6-500 and of a repeated dose (RD) of AVB-S6-500 given intravenously at a single dose level (5 mg/kg) for a total of four doses administered once weekly is evaluated in healthy patients. The SAD and RD pharmacokinetics (PK) and pharmacodynamics (PD) are characterized.

Eligible subjects were randomly assigned in a 3:1 ratio to receive either AVB-S6-500 or placebo at the dose levels indicated in Table 2 and Table 3 and were blinded to treatment.

TABLE 2
Dose Levels for Single Ascending Dose Cohorts

| | |
|---|---|
| | AVB-S6-500/Placebo |
| AVB-S6-500 Dose Level | (n/n) |

| | | |
|---|---|---|
| 1 | mg/kg | 6/2 |
| 2.5 | mg/kg | 6/2 |
| 5 | mg/kg | 6/2 |
| 10 | mg/kg | 6/2 |

TABLE 3
Dose Level for Repeat Dose Cohort

| | AVB-S6-500/Placebo |
|---|---|
| AVB-S6-500 Dose Level | (n/n) |
| 5 mg/kg | 6/2 |

For each subject, the study consisted of 3 periods: a pretreatment period (including a Screening Visit to occur up to 28 days prior to Day 1), a treatment period, and a follow-up period (End-of-Study/Early Withdrawal visit). Following completion of the End-of-Study (EOS)/Early Withdrawal (EW) visit for each cohort, the available safety data from all dose levels are reviewed by the Sponsor, medical monitor (MM), and Investigator to determine whether to proceed with enrollment of the next highest dose of AVB-S6-500.

Subjects enrolled in the single ascending dose cohorts were assigned to receive a single dose of AVB-S6-500 or placebo according to the randomization schedule; subjects in the repeat dosing cohort were assigned to receive four doses of AVB-S6-500 or placebo (administered weekly over 4 weeks) according to the randomization schedule. RD subjects will return to the clinic for administration of each dose of study drug on Day 1 of Week 2 and 3 and continue outpatient visits after each weekly dose administration. Subjects were admitted to the CRU on Day 1 of Week 4 and stay in the CRU for 24 hours after administration of their Week 4 dose to facilitate collection of blood for PK/PD assessments. On Day 2 of Week 4, subjects were discharged from the CRU after completion of all scheduled assessments for that day and continue visits on an outpatient basis through the EOS/EW visit.

All doses of the study drug were prepared as solutions for infusion in 150 ml of diluent (in 250 ml bags) to be administered by intravenous infusion over 1 hour. All treatments were administered in the clinic, by clinic staff. The study drug was provided in vials containing 10 mL of AVB-S6-500 (concentration 20 mg/mL; total AVB-S6-500 content is 200 mg per vial). AVB-S6-500 is not packaged for individual subject numbers. Based on the randomization codes, the pharmacist or properly trained designee will prepare the study drug for intravenous administration. AVB-S6-500 solution for infusion were packaged and labeled according to current Good Manufacturing Practices and supplied to the clinical site in 20 mL-vials (containing 10 mL in each vial).

Blood samples (serum) for analysis of AVB-S6-500 concentration and GAS6 (comprising the pharmacodynamic marker) levels were collected from subjects enrolled in the single ascending cohorts at the following time points relative to dosing: within 45 minutes before dosing (0 hour) and at approximately 1, 2, 4, 6, 8, 24, 72, 120, 168, and 336 hours after dosing. For subjects enrolled in the repeat dose cohort, serum samples for AVB-S6-500 and GAS6 analysis were collected at the following time points: Study week 1 - within 45 minutes before dosing (pre-dose), and at approximately 1, 2, 4, 6, 8, 24, 72, and 120 hours post-dose; Study week 2 - prior to dosing (within 45 minutes of dosing; also serves as 168h time point for Week 1); Study week 3 - prior to dosing (within 45 minutes of dosing); Study week 4-within 45 minutes before dosing (pre-dose), and at approximately 1, 2, 4, 6, 8, 24, 72, 120, 168, 504, 528 and 696 hours post-dose.

For pharmacokinetic and pharmacodynamic analyses, blood samples (4 mL) are collected into serum separator tubes and processed as described in the PK/PD/ADA laboratory manual. Serum samples for the pharmacokinetic assessment of AVB-S6-500 and pharmacodynamic assessment of GAS6 levels are sent to SNBL. One set of serum samples from each subject are shipped on an ongoing basis with multiple shipments per cohort (shipment schedule to be defined in study PK/PD manual). The remaining duplicate or triplicate samples are stored at the CRU as a backup. Blood samples (4 mL) collected at a subset of time points for PK and PD analysis are analyzed for the presence of anti-drug antibodies (ADA). Pharmacokinetic data will be taken into consideration at the point of escalation to the 10 mg/kg dose level. Specifically, PK data was collected following the early dose cohorts and compared to current estimates of human PK using scaling of nonclinical species. These data were then used to reassess safety margins approximated for the subsequent dose levels, as needed.

AVB-S6-500 was well tolerated across all doses. There were no serious adverse events. There were no treatment-related changes noted in physical examinations or vital signs. None of the AEs based on laboratory values were deemed clinically significant, none required treatment and all were asymptomatic. As per protocol, all laboratory values that met CTCAE v 4.03 criteria for subjects given active drug were considered possibly related. None were considered probably/likely or certainly related.

Following single IV infusions, the PK of AVB-S6-500 displayed characteristics similar to other protein therapeutics such as monoclonal antibodies, displaying generally small volumes of distribution and biphasic elimination. The maximal serum AVB-S6-500 concentration (Cmax) and area under the concentration versus time curve (AUC) increased with increasing dose. The increase in Cmax was approximately proportional across this dose range, while the increase in AUC was slightly greater than proportional with dose, suggesting nonlinear elimination kinetics consistent with TMDD. With repeat weekly dosing, the increase in the last measured concentration immediately prior to the subsequent infusion (Ctrough) was approximately 2-fold between doses 1 and 4, suggesting modest accumulation in agreement with the single dose mean half-life of 59 hours. Among all single and repeat dose cohorts, no subjects tested positive for anti-AVB-S6 antibodies. Further, the prolonged suppression of serum GAS6 during repeat administration of AVB-S6 was reflective of the lack of any subjects testing positive for anti-AVB-S6 antibodies.

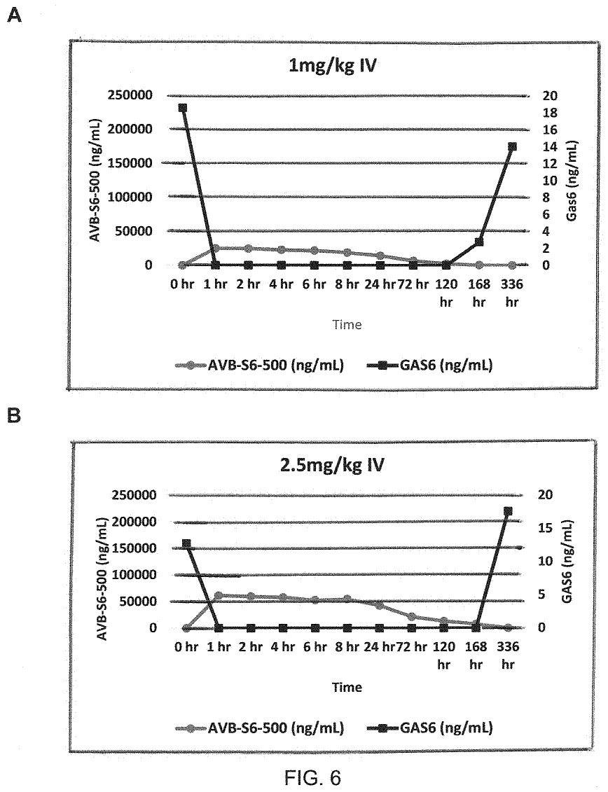

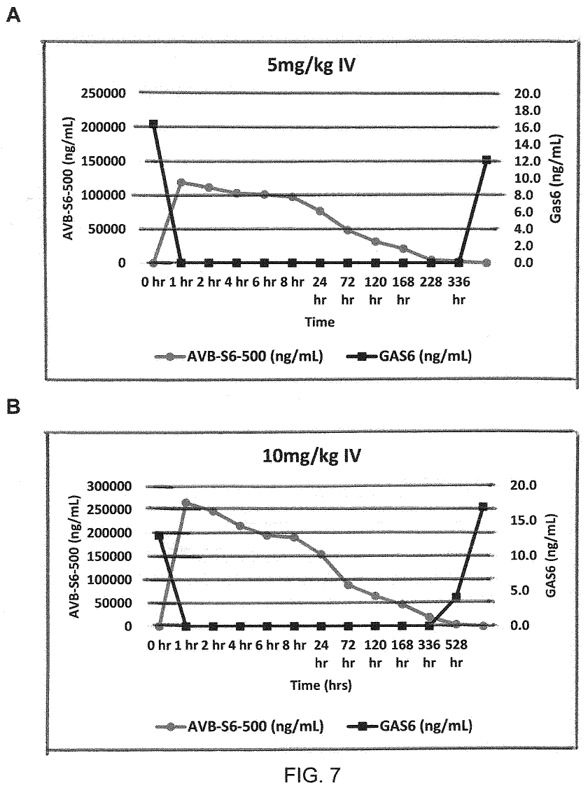

As depicted in FIGS. 6 and 7, serum GAS6 levels were suppressed at one week post dose at 1 mg/kg (observed in ⁴/₆ subjects) (FIG. 6A), one week post dose at 2.5 mg/kg (observed in 6/6 subjects) (FIG. 6B), two weeks post dose at 5 mg/kg (observed in 6/6 subjects) (FIG. 7A) and two weeks post dose at 10 mg/kg (observed in 6/6 subjects) and three weeks post dose at 10 mg/kg (observed in 3/6 subjects.

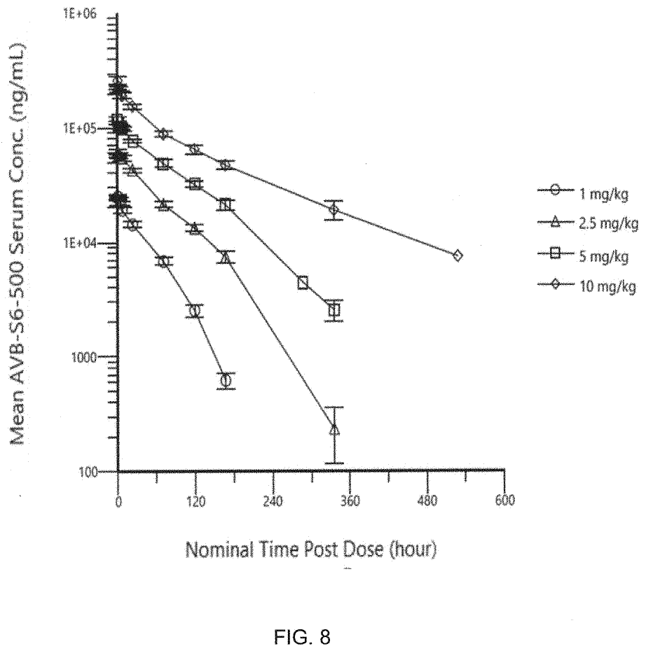

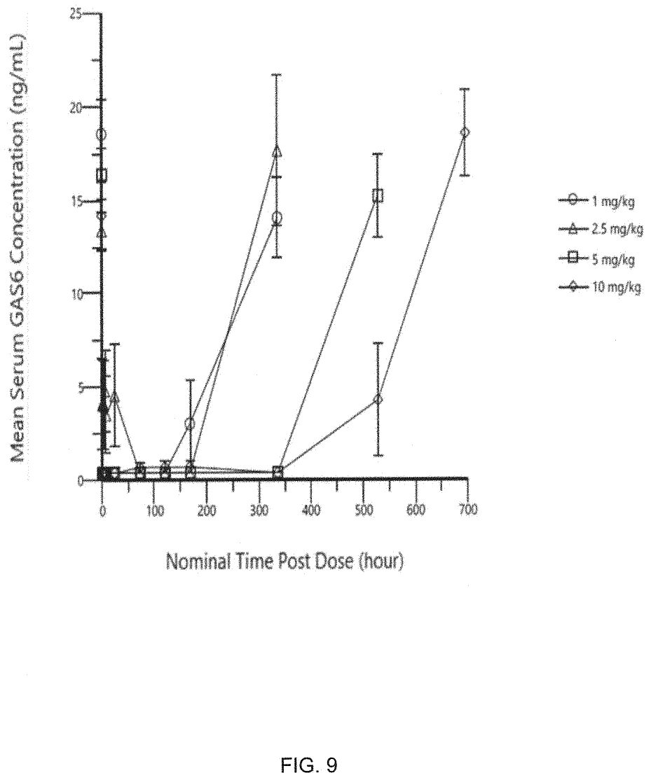

As depicted in FIGS. 8 and 9, AVB-S6-500 drug levels demonstrate a dose response and the lowest dose (1 mg/kg) of AVB-S6-500 is pharmacologically active. The average serum GAS6 levels across subjects was 15.7±3.9 ng/mL. A single infusion of 1, 2.5, 5, or 10 mg/kg AVB-S6 in healthy subjects resulted in an immediate maximal reduction in circulating serum GAS6 concentrations to BLQ levels (2ng/mL). Suppression of GAS6 was maintained for 7 days post infusions of 1 and 2.5 mg/kg AVB-S6-500. Serum GAS6 remained suppressed to below detectable levels for 22 and 29 days post infusion of the 5 and 10 mg/kg doses, respectively. Weekly infusions of 5 mg/kg AVB-S6-500 in healthy subjects resulted in an immediate and sustained maximal reduction in circulating serum GAS6 concentrations to BLQ levels. Suppression of GAS6 was maintained at BLQ levels in all subjects until 504 hours following the final infusion, when GAS6 was measurable above the LLOQ in 2 out of 6 subjects yet had not returned to baseline levels. The GAS6 concentrations remained BLQ in all other subjects (⁴/₆). PK/PD-modeling thus confirmed selection of dosing regimens for cancer studies that would suppress (>90% reduction) sGAS6 and be compatible with chemotherapeutic dosing regimens.

PK/PD profile established in humans was consistent with preclinical data and modeling.

### Example 8

### A Phase 1 b/2 Study of AVB-S6-500 in Combination with Pegylated Liposomal Doxorubicin (PLD) or Paclitaxel in Patients with Platinum-resistant Recurrent Ovarian Cancer

Analysis of sGAS6 levels from 48 patients who had ovarian cancer suggested levels were 2-fold higher than those from the normal healthy volunteer study. Utilizing the monkey PK/PD modeling and Phase 1 healthy volunteer data and simulating increases in sGAS6, suggested that dosing regimens of 5mg/kg every week or 10mg/kg every other week would abrogate sGAS6 levels in cancer patients. PK/PD-modeling confirmed dose selection for ovarian study that would suppress target in >90% of pts and identified multiple dosing regimens that were compatible with chemotherapeutic dosing regimens and limited number of patient visits. Use of proprietary PD assay in combination with establishing the human safety and PK/PD profile in healthy volunteers streamlined the clinical program, guiding dose selection for oncology studies.

### Phase 1 b

In the Phase 1 b portion, the safety and tolerability in an open-label fashion of AVB-S6-500 in combination with pegylated liposomal doxorubicin (PLD) or paclitaxel will be evaluated in platinum-resistant recurrent ovarian cancer patients.

In the AVB-S6-500+PLD patients, AVB-S6-500 will be given as IV infusion over 30 or 60 minutes at a dose of 10 mg/kg in combination with PLD given as IV infusion over 60 minutes at a dose of 40 mg/m² on Day 1 of the 1^{st} treatment cycle. Subsequent doses of 10 mg/kg of AVB-S6-500 will be given every 14 days, starting on Day 15 of the 1St cycle. If 10 mg/kg every 2 weeks (q2w) AVB-S6-500 is not well-tolerated in combination with PLD, the AVB-S6-500 dose will be lowered to 5 mg/kg weekly. Six new patients will be enrolled into this cohort.

In the AVB-S6-500 +paclitaxel patients, AVB-S6-500 will be given weekly as IV infusion over 30 or 60 minutes at 10 mg/kg in combination with paclitaxel given weekly as IV infusion over 60 minutes at 80 mg/m² on days D1, D8, D15 and D22 of every 28-day treatment cycle. In each cohort, six patients will be initially dosed with each combination chemotherapy regimen to evaluate the safety of the combination. If 10 mg/kg AVB-S6-500 is not well-tolerated in combination with paclitaxel, the AVB-S6-500 dose will be lowered to 5 mg/kg weekly. Six new patients will be enrolled into this cohort. If the 5mg/kg dose in combination with paclitaxel is well-tolerated, an additional 6 patients will be enrolled into this dosing regimen.

The RP2D will be a dose/dosing regimen of AVB-S6-500 that is deemed safe/tolerable in combination with the respective chemotherapy backbone and, based on evaluation of 1-month PK/PD from the P1b study, is achieving AVB-S6-500 serum levels >3720 ng/mL and is suppressing serum GAS6 in all patients to BLQ throughout the dosing interval. Treatment with Study Drug will continue until there is no residual tumor (with chemotherapy agent; AVB-S6-500 should be continued for at least a year after a complete response), or until disease progression, death, informed consent withdrawal or unacceptable toxicity.

### Phase 2

After establishing RP2D for AVB-S6-500 and a tolerated combination dose that has the desired effect of abrogating serum GAS6, the Phase 2 portion will compare Progression Free Survival (PFS) in platinum-resistant recurrent ovarian cancer patients treated with AVB-S6-500+PLD versus Placebo+PLD or AVB-S6-500 +paclitaxel versus Placebo +paclitaxel in a randomized, double-blinded fashion. Objective response rate (ORR may also be evaluated as a 2^{nd} endpoint). AVB-S6-500 given as IV infusion over 60 minutes at the RP2D regimen, starting on Day 1, for a 28-day treatment cycle. Physician's choice of chemotherapy includes the following options: 1) Paclitaxel given weekly as IV infusion over 60 minutes at a dose of 80 mg/m² for a 28-day treatment cycle or 2) PLD is given as IV infusion over 60 minutes at a dose of 40 mg/m² on Day 1 of a 28-day treatment cycle. Treatment with Study Drug will continue until there is no residual tumor (with chemotherapy agent; AVB-S6-500 should be continued for at least a year after a complete response), or until disease progression, death, informed consent withdrawal or unacceptable toxicity.

Patients will be enrolled and randomized 2:1 into one of the two treatment arms: Arm A (AVB-S6-500+physician's choice chemotherapy) and Arm B (Placebo +physician's choice chemotherapy). Each arm has two cohorts, one for PLD combination regimen and one for Pac combination regimen.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### Sequence Listings

The nucleic and amino acid sequences listed in the accompanying sequence listing are shown using standard letter abbreviations for nucleotide bases and three letter code for amino acids, as defined in 37 C.F.R. 1.822.

SEQ ID NO: 1 is the amino acid sequence of a human AXL polypeptide.

Human AXL polypeptide amino acid sequence
SEQ ID NO: 1
MGRVPLAWCLALCGWACMAPRGTQAEESPFVGNPGNITGARGLTGTLRCQL
QVQGEPPEVHWLRDGQILELADSTQTQVPLGEDEQDDWIVVSQLRITSLQL
SDTGQYQCLVFLGHQTFVSQPGYVGLEGLPYFLEEPEDRTVAANTPFNLSC
QAQGPPEPVDLLWLQDAVPLATAPGHGPQRSLHVPGLNKISSFSCEAHNAK
GVTTSRTATITVLPQQPRNLHLVSRQPTELEVAWTPGLSGIYPLTHCTLQA
VLSNDGMGIQAGEPDPPEEPLTSQASVPPHQLRLGSLHPHTPYHIRVACTS
SQGPSSWTHWLPVETPEGVPLGPPENISATRNGSQAFVHWQEPRAPLQGTL
LGYRLAYQGQDTPEVLMDIGLRQEVTLELQGDGSVSNLTVCVAAYTAAGDG
PWSLPVPLEAWRPGQAQPVHQLVKEPSTPAFSWPWWYVLLGAVVAAACVLI
LALFLVHRRKKETRYGEVFEPTVERGELVVRYRVRKSYSRRTTEATLNSLG
ISEELKEKLRDVMVDRHKVALGKTLGEGEFGAVMEGQLNQDDSILKVAVKT
MKIAICTRSELEDFLSEAVCMKEFDHPNVMRLIGVCFQGSERESFPAPVVI
LPFMKHGDLHSFLLYSRLGDQPVYLPTQMLVKFMADIASGMEYLSTKRFIH
RDLAARNCMLNENMSVCVADFGLSKKIYNGDYYRQGRIAKMPVKWIAIESL
ADRVYTSKSDVWSFGVTMWEIATRGQTPYPGVENSEIYDYLRQGNRLKQPA
DCLDGLYALMSRCWELNPQDRPSFTELREDLENTLKALPPAQEPDEILYVN
MDEGGGYPEPPGAAGGADPPTQPDPKDSCSCLTAAEVHPAGRYVLCPSTTP
SPAQPADRGSPAAPGQEDGA

1-29. (canceled)

30. A method for treating a human metastatic cancer in a human patient comprising administering to said patient a therapeutically effective dose of a soluble AXL variant polypeptide according to a regimen determined to achieve improved progression-free survival (PFS) as compared to control, wherein the soluble AXL variant polypeptide lacks the AXL transmembrane domain; lacks a functional fibronectin (FN) domain; has one or more than one Ig1 domain and, optionally, one or more than one Ig2 domain; and has a set of amino acid modifications of the wild-type AXL sequence (SEQ ID NO:1), selected from the group consisting of:
1) Gly32Ser, Asp87Gly, Va192Ala, and Glyl27Arg,
2) Glu26Gly, Va179Met, Va192Ala, and Glyl27Glu; and
3) Gly32Ser, Ala72Val, Asp87Gly, Va192Ala, and Glyl27Arg;
wherein said modification increases the affinity of the AXL polypeptide binding to Growth arrest-specific protein 6 (GAS6), and wherein the soluble AXL variant polypeptide is fused to an Fc region.

31. A method according to claim 30, wherein the human metastatic cancer overexpresses the biomarker GAS6 and/or AXL.

32. A method according to claim 30, wherein the human metastatic cancer is a recurrent cancer.

33. A method according to claim 30, wherein the human metastatic cancer is resistant to standard therapies.

34. A method according to claim 30, wherein the human metastatic cancer is a chemoresistant cancer.

35. A method according to claim 30, wherein the human metastatic cancer is a platinum resistant cancer.

36. A method according to claim 30, wherein the human metastatic cancer is selected from the group consisting of B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer;
lymphomas; sarcomas; multiple myeloma; and leukemias.

37. A method according to claim 36, wherein the human metastatic cancer is ovarian cancer.

38. A method according to claim 36, wherein the human metastatic cancer is breast cancer.

39. A method according to claim 36, wherein the human metastatic cancer is uterine cancer.

40. A method according to claim 30, wherein the soluble AXL variant is administered in combination with a second therapy selected from the group consisting of cytoreductive therapy, therapy using a chemotherapeutic agent, therapy using a poly(ADP-ribose) polymerase (PARP) inhibitor, and immunotherapy, wherein the combination has a synergistic effect.

41. A method according to claim 40, wherein the chemotherapeutic agent is selected from the group consisting of: daunorubicin, adriamycin (doxorubicin), epirubicin, idarubicin, anamycin, MEN 10755, etoposide, teniposide, vinblastine, vincristine, vinorelbine (NAVELBINE); vindesine, vindoline, vincamine, mechlorethamine, cyclophosphamide, melphalan (L-sarcolysin), carmustine (BCNU), lomustine (CCNU), semustine (methyl-CCNU), streptozocin, chlorozotocin, cytarabine (CYTOSAR-U), cytosine arabinoside, fluorouracil (5-FU), floxuridine (FUdR), thioguanine (6-thioguanine), mercaptopurine (6-MP), pentostatin, fluorouracil (5-FU), methotrexate, 10-propargyl-5,8-dideazafolate (PDDF, CB3717), 5,8-dideazatetrahydrofolic acid (DDATHF), leucovorin, cisplatin (cis-DDP), carboplatin, oxaliplatin, hydroxyurea, gemcitabine, and N-methylhydrazine.

42. A method according to claim 40, wherein the immunotherapy is selected from the group consisting of: treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

43. A method according to claim 40, wherein the PARP inhibitor is selected from the group consisting of: ABT-767, AZD 2461, BGB-290, BGP 15, CEP 9722, E7016, E7449, fluzoparib, INO1001, JPI 289, MP 124, niraparib, olaparib, ONO2231, rucaparib, SC 101914, talazoparib, veliparib, WW 46, or salts or derivatives thereof.

44. A method according to claim 30, wherein the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, and of about 20.0 mg/kg.

45. A method according to claim 44, wherein the dose is given weekly.

46. A method according to claim 44, wherein the dose is given biweekly.

47. A method according to claim 44, wherein the dose is 5 mg/kg given weekly.

48. A method according to claim 44, wherein the dose is 10 mg/kg given weekly.

49. A method according to claim 44, wherein the dose is 20 mg/kg given bi-weekly.
